(19) United States
(12) Patent Application Publication
(10) Pub. No.: US 2011/0195029 A1
(43) Pub. Date: Aug. 11, 2011

(54) FLUORESCENT EMULSION

(75) Inventors: Laurent Guyon, Grenoble (FR); Mathieu Goutayer, St. Malo (FR); Fabrice Navarro Y Garcia, Fontaine (FR); Isabelle Texier-Nogues, Grenoble (FR)

(73) Assignee: COMMISSARIAT A L'ENERGIE ATOMIQUE ET AUX ENE ALT, Paris (FR)

(21) Appl. No.: 13/058,984

(22) PCT Filed: Aug. 11, 2009
(86) PCT No.: PCT/IB2009/006766

§ 371 Date: Apr. 28, 2011

(30) Foreign Application Priority Data
Aug. 14, 2008 (FR) 08 04601

(51) Int. Cl. A61K 49/00

(52) U.S. Cl. 424/9.6

(57) ABSTRACT

The invention relates to a fluorescent emulsion, to its uses and to labelling reagents comprising it.

The fluorescent emulsion of the invention is of the oil-in-water type, comprising at least one aqueous continuous phase in which droplets of at least one oil phase are dispersed, said oil phase droplets being stabilized by a surfactant layer, characterized in that it comprises at least one pair of labels, differing from one another, formed from a donor fluorescent label that absorbs at a wavelength λ₁ and emits at a wavelength λ₂, different from λ₁, and an acceptor label that absorbs at the emission wavelength λ₂ of the donor fluorescent label; in that the donor fluorescent label and the acceptor label are kept close together by the encapsulation of one of them in the oil phase droplets and either by linking the other of them to the oil phase droplet/aqueous phase interface, or by the encapsulation of the other of them in the oil phase droplets; and in that it comprises molecules of at least one amphiphilic surfactant and at least one solubilizing lipid.

The fluorescent emulsion of the invention is applicable in the field of optical fluorescence imaging, in particular optical fluorescence biomedical imaging.

The invention relates to a fluorescent emulsion, to its uses and to labelling reagents comprising it.

Optical imaging techniques based on the exploitation of the diffuse component of the detected signal are being developed further and further as they allow scattering objects, and more specifically thick scattering objects, to be probed.

In the field of biomedical imaging, these techniques offer an alternative to the conventional techniques: radiography and X-ray tomography, positron emission tomography and magnetic resonance imaging for the detection and location, for example by diffusive optical tomography, of for example cancerous tumours. In these techniques, the wavelength range in the visible of non-ionizing radiation, more precisely in the red or near infrared in which biological tissues have an absorption minimum, is used to detect the presence of an abnormally absorbent and/or scattering region.

More recently, optical fluorescence molecular imaging techniques are being developed further and further thanks to the use of specific fluorescent labels. These are preferably attached to the target cells of interest, for example cancerous cells, and offer better detection contrast than non-specific labels. The purpose of these techniques is not only to locate the fluorescent labels in space but also to determine the concentration thereof, thereby making it possible, indirectly, to locate the tumour and obtain information about its shape and also its biological activity.

Instruments using the propagation of light into a scattering medium may be divided into three categories depending on the light source used: continuous; frequency-modulated; and time-modulated. Historically, instruments using a continuous light source were the first to be developed. However, they have the drawback, among other things, of not providing information about the scattering properties of the tissue, which therefore have to be provided a priori. The time-modulated and frequency-modulated approaches are related by a Fourier transform and are both richer in information. A single acquisition using a source/detector pair makes it possible for example to measure the optical absorption and isotropic scattering properties, denoted respectively by μₐ and μₛ, of a homogeneous medium.

In optical fluorescence molecular imaging techniques, fluorescent labels or fluorescent contrast agents are injected into the scattering tissue to be studied and are localized, either specifically or non-specifically, in the region to be studied. The tissue is then illuminated with a quasi-monochromatic light source obtained using a band-pass filter or a low-pass filter, or else a laser beam. The light from the source is scattered in the tissue and some of the photons reach the fluorescent label or fluorophore molecules, which re-emit the energy provided by the light source that they absorb, by fluorescent emission at a wavelength shifted towards the red. The light emitted by the fluorophore itself propagates into the scattering tissue to be studied until reaching the edges and emerging therefrom. It is this output light which is collected by an imaging device, such as a camera, an intensified camera, optical fibres or another imaging device, filtered beforehand so as to cut off the excitation signal and collect only the fluorescence photons.

The wavelengths of the light emitted by the source and by the fluorescence re-emitted by the fluorophore lie, for human and animal tissue, in the red or the near infrared, called the therapeutic window region, as it is at these wavelengths that human and animal tissue absorbs the least. Specifically, blood is responsible for the absorption of light at shorter wavelengths and water at longer wavelengths.

The fluorescent molecules, or fluorophores, are therefore chosen to absorb and emit in these wavelengths, i.e. between 640 and 900 nm.

At the present time, fluorescent molecules are not injected as such into the tissue to be studied, but in the form of an optical probe. In general, this optical probe is a molecular assembly consisting of the fluorescent molecule which may, for example, be an organic fluorophore, a lanthanide complex, or else a luminescent semiconductive nanocrystal (“quantum dot”, such as CdSe, CdTe, InP, Si, etc.).

These optical probes may also comprise one or more of the following components:
a) a biological ligand, which makes it possible to image a specific biological process. Such a ligand may be:
i) a biological targeting ligand: it is then a biological entity (antibody, peptide, saccharide, etc.) or a chemical entity (folic acid, for example) which enables specific recognition of certain cells (for example, of tumour cells, as described for example in the article by S. Achilefu, Technology in Cancer Research & Treatment, 2004, 3, 393-408) or of certain organs,
ii) a biological ligand which is a label for a given biological activity, for example, an enzymatic activity. For example, these biological ligands will be a peptide that can be cleaved by a given protease, onto the end of which an inhibitor of the fluorescence of the label will be grafted. Ligands of this type make it possible to specifically image the enzymatic activity of the protease, as is reported in the article by C. H. Tung, Biopolymers, 2004, 76, 391-403. Another example consists of a biological ligand comprising a disulphide bridge separating the label from an inhibitor of the fluorescence of said label. This biological ligand then makes it possible to specifically image the internalization of the optical probe in a cell, as described, for example, in the French patent application published under the number FR 2 888 938;
b) a stealth agent: this is an entity which is added to the optical probe in order to confer on it stealth with respect to the immune system, to increase its circulation time in the organism, and to slow down its elimination;
c) an “assembly vector”: this is an entity which can make it possible to assemble the fluorescent label(s) and/or the biological targeting ligand(s) and/or the stealth agent(s) and/or one or more other functionalities (drug delivery, other imaging mode, therapeutic function, for example).

The fluorescent molecules may also be included in emulsions.

However, human or animal tissue without a fluorescent label has an intrinsic fluorescence, called endogenous fluorescence or auto-fluorescence, which adds a spurious signal. Inelastic scattering of the excitation or poorly filtered excitation may also be sources of spurious signals.

In addition, the fluorescence re-emitted by the fluorescent molecule has a wide spectrum. It is therefore difficult to have signals from different fluorescent molecules, also called hereafter donor fluorescent labels, simultaneously, which means little or no multiplexing is possible.

Finally, at the present time, none of the existing optical probes can determine when the fluorescent molecule (the fluorescent label) is delivered into the organism.

Another field of application of optical fluorescence imaging is for monitoring the delivery, the variation in shape, size or state, of a substance of interest in a host medium.

It may for example be the monitoring of the delivery of a drug in a human or animal or the delivery of a pesticide in a plant or in a cell or tissue, or a particular organ of this human, animal or plant, or else a synthetic medium representative of these cells, tissues or organs.

However, the host medium may also be a synthetic or natural medium containing an organism or a particular substance, the path of which it is desired to monitor. However, optical fluorescence imaging may also be used to study nanoemulsions in order to monitor the evolution in size of nanoparticles or to know when these nanoparticles burst or to determine the rate of release of a label.

The object of the invention is therefore to provide a fluorescent emulsion that can be used in all these applications and can inhibit and even suppress the spurious signal due to the auto-fluorescence of the host medium into which said emulsion is injected and/or allow the simultaneous use of several fluorescent labels and/or monitor the delivery, change of shape or size, etc. of a drug or a substance of interest in a host medium.

For this purpose, the invention provides a fluorescent emulsion of the oil-in-water type, comprising an aqueous continuous phase in which droplets of an oil phase are dispersed, said droplets being stabilized by a surfactant layer, characterized in that it comprises at least one pair of labels, differing from one another, formed from a donor fluorescent label that absorbs at a wavelength λ₁ and emits at a wavelength λ₂, different from λ₁, and an acceptor label that absorbs at the emission wavelength λ₂ of the donor fluorescent label; in that the donor fluorescent label and the acceptor label are kept close together by the encapsulation of one of them in the oil phase droplets and either by linking the other of them to the oil phase droplet/aqueous phase interface, or by the encapsulation of the other of them in the oil phase droplets; and in that it comprises molecules of at least one amphiphilic surfactant and molecules of at least one solubilizing lipid.

In a first preferred embodiment of the fluorescent emulsion of the invention, the acceptor label re-emits the light energy emitted by the donor fluorescent label in the form of light energy having a wavelength λ₃, which differs from the wavelengths λ₁ and λ₂.

In a second preferred embodiment of the fluorescent emulsion of the invention, the acceptor label re-emits no or little light energy provided by the donor label in the form of light energy.

In all the embodiments of the fluorescent emulsion of the invention, the wavelengths λ₁, λ₂ and λ₃ are between 640 and 900 nm inclusive.

Also preferably, in all the embodiments of the fluorescent emulsion according to the invention, the oil phase droplets have an average diameter of between 10 and 200 nm inclusive.

In a first variant of all the embodiments of the fluorescent emulsion of the invention, the donor fluorescent label and the acceptor label are, each independently of the other, lipophilic or amphiphilic and are kept close together by encapsulation in the oil phase droplets.

In this first variant, and in a first preferred embodiment, the donor fluorescent label is 1,1′-dioctadecyl-3,3,3′,3′-tetramethylindodicarbocyanine perchlorate (DiD) and the acceptor label is 1,1′-dioctadecyl-3,3,3′,3′-tetramethylindotricarbocyanine iodide (DiR).

In another preferred embodiment of this first variant, the donor fluorescent label is 1,1′-dioctadecyl-3,3,3′,3′-tetramethylindotricarbocyanine iodide and the acceptor label is indocyanine green (ICG).

In a second variant of all the embodiments of the fluorescent emulsion of the invention, either the donor label or the acceptor label is amphiphilic and tied to the oil phase droplet/aqueous phase interface by being linked directly to the membrane of the oil phase droplets, the other being encapsulated in the oil phase droplets.

In a third variant of all the embodiments of the fluorescent emulsion of the invention, either the donor fluorescent label or the acceptor label is tied to the oil phase droplet/aqueous phase interface by linking to the surfactant molecules, the other being encapsulated in the oil phase droplets.

In this variant and in a first embodiment, the label linked to the surfactant molecules is linked to these surfactant molecules by a covalent bond.

In a second embodiment of this variant, the label linked to the surfactant molecules is linked to these surfactant molecules by a disulphide bridge or a peptide bridge or a hydrazone bond.

The invention also proposes the use of a fluorescent emulsion according to the invention for the manufacture of a labelling reagent for monitoring the delivery of a drug or a substance of interest in a host medium.

The invention also proposes a labelling reagent for monitoring the delivery of a drug or a substance of interest in a host medium comprising an emulsion according to the invention and a drug or a substance of interest encapsulated in the oil phase droplets.

The invention also proposes a labelling reagent for monitoring the delivery of a drug or a substance of interest in a host medium comprising a fluorescent emulsion according to the invention and a drug or a substance of interest linked to either the donor fluorescent label or to the acceptor label.

Finally, the invention proposes the use of an emulsion according to the invention for the manufacture of a labelling reagent for optical fluorescence imaging.

The invention will be better understood and other characteristic advantages thereof will become more clearly apparent on reading the following explanatory description.

An emulsion is a mixture of two immiscible liquid substances, made up of a continuous phase and a dispersed phase. One substance is dispersed in the second substance (the continuous phase) in the form of droplets (the dispersed phase). The mixture remains stable by virtue of the action of amphiphilic molecules, called emulsifiers or surfactants, which lie at the interface between the two phases. Emulsions are metastable supramolecular structures. These structures are to be distinguished from polmersomes and micelles.

Polymersomes (a family comprising liposomes) are vesicles of a few tens to a few thousand nm in diameter. These vesicles are composed of one or more bilayers of surfactants which make(s) it possible to separate the intravesicular medium from the external medium, the two media being of the same (aqueous) nature.

Micelles consist of self-assembled surfactant aggregates, a few nanometres in diameter. The surfactants are organized in such a way as to direct their hydrophilic part towards the outside (the solvent) and their hydrophobic chains towards the core of the micelle.

Emulsions have already been used for the manufacture of contrast agents. In all these emulsions, a label is introduced so as to allow display via the desired technique.

Thus, Patent Application US 2005/00079131 describes emulsions of the oil-in-water type in which the oil droplets have average diameters between 10 and 200 nm, which corresponds to the generally accepted definition of the terms nanoemulsion, or miniemulsion or ultrafine emulsion, or else submicron emulsion, in which a fluorophore is present within the surfactant layer surrounding the oil droplets and enabling the emulsion to be stabilized. Apart from the fact that in this patent application the fluorophore is an auxiliary imaging agent, the main imaging agent being an element having a high atomic number (Z), this fluorophore is not used in combination with another label that absorbs the light energy emitted by the fluorophore.

Now, to solve the problems of spurious fluorescent signals due to the auto-fluorescence of human and animal tissue, to allow the simultaneous use of various fluorescent labels, or else to make it possible to determine when and if a product, for example a drug, is delivered in a host material, the invention uses not only a fluorophore, called hereafter a donor fluorescent label, but also an acceptor label, the donor label transferring its light energy to the acceptor label, which acceptor label will restore this energy either in the form of a fluorescence, but at a different wavelength from the emission wavelength of the fluorescence of the donor fluorescent label, or in the form of non-luminous energy, for example in the form of thermal energy.

More precisely, to solve the abovementioned problems, the invention is based on the phenomenon of fluorescence resonance energy transfer, called FRET or RET. This energy transfer is a non-radiative process in which a donor fluorescent label in the excited state transmits its fluorescence energy to an acceptor label placed in the immediate vicinity (a few nanometers therefrom). When the acceptor label is itself a fluorophore, it re-emits the energy transferred by the donor fluorescent label also in the form of a fluorescence. In this case, FRET has in particular the effect of reducing the fluorescence of the donor fluorescent label and increasing that of the acceptor, and also of modifying the fluorescence wavelength for read-out by optical fluorescence imaging. FRET also has the effect of modifying the lifetime of the fluorescences.

The change in fluorescence wavelength of the donor fluorescent label is considerable, in particular in optical fluorescence imaging of human and animal tissue. Specifically, as already mentioned, in the case of human and animal tissue, the fluorescence must take place in the red or the near infrared, in which wavelength range tissue without a donor fluorescent label also has an intrinsic fluorescence. Thanks to the use of the acceptor label, the fluorescence is shifted towards the red. Furthermore, it is possible to filter the fluorescence which is shifted into wavelength ranges (again in the red or near infrared) in which the spurious fluorescence of the tissue is either greatly reduced, or even absent. The fluorescence of the acceptor label may then be filtered with band-pass and/or high-pass filters and shifted into a range in which the spurious fluorescent signal of the tissue is much weaker. Thus, the desired signal/spurious signal ratio is increased.

However, the acceptor label may also be what is called a “quencher”, i.e. a label that absorbs the transmitted light energy transferred by the donor fluorescent label but does not re-emit this energy in the form of fluorescence light energy. In fact, it absorbs the light energy and restores it in another form of energy, for example thermal energy. In this case, the fluorescence of the donor fluorescent label is, if not completely stopped, in any case greatly inhibited, and what will be detected is the reappearance of the fluorescence of the donor fluorescent label.

The phenomenon of energy transfer used in the invention takes place when two conditions are fulfilled:
1) when the acceptor label absorbs in the range of wavelengths emitted by the donor fluorescent label; and
2) when the donor fluorescent label and the acceptor label are close to each other, but without being directly linked together.

By introducing the donor fluorescent label and the acceptor label into an emulsion, it is possible to meet the second condition, in that it makes it possible, in a first embodiment, to encapsulate the donor fluorescent label and the acceptor label in the oil (oil phase) droplets, thereby enabling them to be kept at a defined distance from each other.

However, only one of the labels may be encapsulated, the other being linked either directly or indirectly to the membrane of the oil droplet in which the other label is encapsulated. Once again, a suitable distance is maintained between the two labels.

In order for both labels to be encapsulated in the oil droplets, these two labels must either be lipophilic, or have been rendered lipophilic by the grafting, for example, of a fatty chain, or else they must be amphiphilic with a high solubility in the oil phase constituting the droplets.

In order for the label to be linked directly to the membrane, said label must be amphiphilic, or have been rendered amphiphilic by the grafting of a lipophilic chain or a hydrophilic chain, depending on its initial lipophilicity or hydrophilicity, and its solubility in the oil phase must not be sufficient to keep it encapsulated in the oil phase droplet. However, the label may be linked to the membrane of the oil droplet via surfactant molecules which are themselves amphiphilic by nature, which are present in the surfactant layer of the emulsion in order to stabilize it. This is an advantage in particular when the label is hydrophilic.

The surfactant molecule may be linked either via a covalent bond or else via, for example, a disulphide bridge, a hydrazone bond or a cleavable bridge. Such a cleavable bridge may be a disulphide bridge, which has been broken and cleaved by a change in redox potential. It may also be a hydrazone bond, which is sensitive to a change in pH, this being moreover an advantage when it is desired to display tumour cells that often have a more acid pH than healthy cells. The cancerous character of the cells is thus revealed, either when the acceptor label is a label which is not itself fluorescent, the fluorescence of the donor fluorescent label then being recovered, or when the acceptor label itself emits a fluorescence, by detecting the fluorescence of the donor fluorescent label and no longer that of the acceptor label. The cleavable bridge may also be a peptide bridge, for example one that can be cleaved by proteases such as metalloproteases, or by cathepsines which may be overexpressed in certain tumour models.

To summarize, any donor fluorescent label/acceptor label pair may be used in the emulsions of the invention, and these labels may be either both positioned inside the oil droplets, or one of them is linked to the membrane of the oil droplet at its external surface, either directly or indirectly, and the other of them is inside the oil droplets, provided that the acceptor label absorbs at the emission wavelength λ₂ of the donor fluorescent label, which itself absorbs at a wavelength λ₁ different from λ₂. The acceptor label may itself by a fluorophore, and in this case it must emit at a wavelength λ₃ different from the wavelengths λ₁ and λ₂. This means that the labels of the pair are different from each other.

Because the emulsion of the invention is more particularly intended to be injected into tissue in a host medium, which is a human or an animal, the donor fluorescent label must absorb and emit in the near-infrared wavelength range, i.e. in the wavelength range lying between 640 nm and 900 nm. Consequently, in that case the acceptor label must itself also absorb in this same wavelength range and, when it is itself fluorescent, it must re-emit in this same wavelength range.

Also preferably, the emulsions used in the invention are nanoemulsions, i.e. oil droplets having a size of between 10 and 200 nm, and more preferably between 10 and 80 nm inclusive, so as to allow internalization of the emulsions in the cells of human or animal tissue.

Within the context of the invention, the term “droplet” encompasses both the actual oil droplets and the solid particles resulting from an emulsion of the oil-in-water type in which the oil used is a crystallizable oil. In this case, such an emulsion is referred to as a solid emulsion.

The oils that can be used are biocompatible oils chosen from natural oils of plant or animal origin, synthetic oils and mixtures thereof. These oils are used without chemical or physical modification prior to the formation of the emulsion.

Among such oils mention may in particular be made of oils of plant origin, among which are in particular soybean oil, palm oil, groundnut oil, olive oil, flax oil, grapeseed oil and sunflower oil; oils of animal origin, among which are in particular fish oils; synthetic oils, among which are in particular triglycerides, diglycerides and monoglycerides; it being possible for said oils to be used alone or as mixtures.

These oils may be first-expression, refined or interesterified oils.

According to one particularly preferred embodiment of the invention, these oils are chosen from oils which are not very water-soluble, i.e. those which have a hydrophilic-lipophilic balance (HLB) generally of less than 8, and even more preferably of between 3 and 6, such as, for example, soybean oil.

According to one preferred embodiment, the oil phase is made up of at least 10% by weight of an oil of which the viscosity is greater than or equal to 100 cP at 20° C. (viscosity values tabulated, for example, in the Handbook of Chemistry and Physics, CRC Press, 88th edition, 2007). The presence of such an oil in the oil phase makes it possible to confer, on the labels formulated in the emulsions, fluorescence lifetimes particularly suitable for in vivo time-resolved fluorescence imaging.

The emulsion comprises surfactants, and in particular at least one amphiphilic surfactant, in order to form the surfactant layer for stabilizing the oil droplets within the emulsion.

These amphiphilic surfactants (comprising a solid part) are generally chosen from compounds of which the lipophilic part comprises a linear or branched, saturated or unsaturated chain containing from 8 to 30 carbon atoms. They may be chosen from phospholipids, cholesterols, lysolipids, sphingomyelins, tocopherols, glucolipids, stearylamines, cardiolipins of natural or synthetic origin; molecules composed of a fatty acid coupled to a hydrophilic group by an ether or ester function, such as sorbitan esters, for instance the sorbitan monooleate and monolaurate sold under the name Span® by the company Sigma; polymerized lipids; lipids conjugated to short chains of polyethylene oxide (PEG) such as the nonionic surfactants sold under the trade names Tween® by the company ICI Americas Inc. and Triton® by the company Union Carbide Corp.; sugar esters, such as sucrose monolaurate and dilaurate, sucrose monopalmitate and dipalmitate, and sucrose monostearate and distearate; it being possible for said surfactants to be used alone or as mixtures.

According to the invention, the amphiphilic surfactant(s) is (are) preferably surfactants that are of natural origin and are assimilable (biocompatible), such as soybean lecithin, phospholipids and cholesterol.

The preferred amphiphilic surfactant in the invention is lecithin.

The emulsion of the invention comprises, in combination with the amphiphilic surfactant, a solubilizing lipid.

The solubilizing lipid allows large amounts of surfactants, in particular the amphiphilic surfactant(s), to be dissolved.

Thus, on the one hand, it allows the preparation of emulsions in which the dispersed phase has a small diameter, i.e. nanoemulsions, when this is desired, and, on the other hand and most particularly, it allows a large number of labels to be dissolved in the emulsion of the invention when the labels are lipophilic or amphiphilic and enables a large number of label molecules to be grafted onto the surfactants, particularly amphiphilic surfactants, as these, being better dissolved, may be present in larger amounts. Therefore, the optical properties of the emulsion are improved.

The solubilizing lipid is a lipid having an affinity with the amphiphilic surfactant sufficient to allow the amphiphilic surfactant to dissolve. When the amphiphilic surfactant is a phospholipid, one appropriate solubilizing lipid is a glycerol derivative and in particular a glyceride obtained by the esterification of glycerol with fatty acids. The solubilizing lipid used is advantageously chosen according to the amphiphilic surfactant used. In general, it will have a similar chemical structure so as to ensure the desired solubilization. It may be an oil or a wax.

The preferred solubilizing lipids, in particular in the case of phospholipids, are glycerides of fatty acids, especially saturated fatty acids, and in particular saturated fatty acids containing 8 to 18 carbon atoms, or more preferably 12 to 18 carbon atoms.

Glycerides of saturated fatty acids, comprising 0% to 20% by weight of C8 fatty acids, 0% to 20% by weight of 010 fatty acids, 10% to 70% by weight of C12 fatty acids, 5% to 30% by weight of C14 fatty acids, 5% to 30% by weight of C16 fatty acids and 5% to 30% by weight of C18 fatty acids, are preferred.

Particularly preferred are the mixtures of semi-synthetic glycerides, which are solid at room temperature, sold under the trade name Suppocire®NC by the company Gattefossé. N-type Suppocire® products are obtained by direct esterification of fatty acids and glycerol. They are semi-synthetic glycerides of C8 to C18 saturated fatty acids, the quail-quantitative composition of which is indicated in the table below.

TABLE
Fatty acid composition of Gattefossé
SuppocireNC ®

| Chain length | % by weight |
|---|---|
| C8 | 0.1 to 0.9 |
| C10 | 0.1 to 0.9 |
| C12 | 25 to 50 |
| C14 | 10 to 24.9 |
| C16 | 10 to 24.9 |
| C18 | 10 to 24.9 |

The amount of solubilizing lipid may vary greatly, depending on the nature and the amount of amphiphilic surfactant present in the oil phase.

As was seen above, the nature of the labels that can be used in the emulsion of the invention is not critical provided that they are compatible with fluorescence imaging and, if they are used on human or plant tissue, provided that they absorb and emit at a wavelength between 640 and 900 nm and that there is spectral exchange between donor fluorescent label emission and acceptor label absorption.

At least one of the labels must be a fluorescent label, i.e. a fluorophore.

Such labels may be fatty acid analogues, sphingolipids, steroids, polysaccharides and phospholipids functionalized with a group which absorbs and emits in the near infrared, and amphiphilic derivatives thereof. Mention may more particularly be made of the derivatives of cyanins, of rhodamines, of fluoresceins, of coumarins, of squaraines, of azulenes, of xanthenes, of oxazines and of 4,4-difluoro-4-bora-3a,4a-diaza-s-indacene (boron dipyrromethene), and also amphiphilic derivatives of said fluorophores.

By way of example, mention may be made more particularly of the products sold under the trade names Bodipy® 665/676 (Ex/Em.) by the company Invitrogen; amphiphilic derivatives of dialkylcarbocyanines, such as the 1,1′-dioctadecyl-3,3,3′,3′-tetramethylindodicarbocyanine perchlorate (DiD) sold under the reference D-307 by the company Invitrogen and the 1,1′-dioctadecyl-3,3,3′,3′-tetramethylindotricarbocyanine iodide (DiR) sold under the reference D-12731 by the company Invitrogen.

According to one preferred embodiment of the invention, the fluorophores are chosen from amphiphilic derivatives of dialkylcarbocyanins.

The acceptor label, when it is itself fluorescent, may be chosen from the same compounds as those mentioned above for the donor fluorescent label, provided that it is compatible with the latter.

When the acceptor label is not itself a fluorescent label, any quencher molecule may be suitable. As an example, mention may be made, in order to inhibit or eliminate the fluorescence of a donor fluorescent label emitting in the near infrared: Dabcyl® and derivatives, and Black Hole Quencher® (BHQ) products such as BHQ-1, BHQ-2 or BHQ-3 (from Biosearch Technologies); Nanogold Particles® (from Nanoprobes); Eclipse Dark Quencher® (from Epoch Bioscience); Elle Quencher® (from Oswell); Cy7Q (from Amersham Biosciences); Fluoquench™ products such as Fluoquench™ 670 and Fluoquench™ 680 (from Interchim); and QSY® dyes, such as QSY®7, QSY®9 and QSY® 21 (from Molecular Probes).

Preferably, the emulsion of the invention includes cosurfactants, in order to improve the stability of the emulsion, and particularly stealth cosurfactants.

Such stealth cosurfactants are preferably amphiphilic molecules of which the hydrophilic part is completely or partially composed of a polyethylene oxide chain (PEO or PEG) and in which the number of PEO units preferably ranges between 2 and 500. The stealth cosurfactants may also be polysaccharide compounds, such as dextrans, for example. By way of example of stealth cosurfactants that can be used according to the present invention, mention in particular may be made of polyethylene glycol/phosphatidylethanolamine (PEG/PE) conjugate compounds, fatty acid ethers of polyethylene glycol, such as the products sold under the trade name Brij® (for example, Brij® 35, 58, 78 or 98) by the company ICI Americas Inc., fatty acid esters of polyethylene glycol, such as the products sold under the trade name Myrj® by the company ICI Americas Inc. (for example, Myrj® 45, 52, 53 or 59), and ethylene oxide/propylene oxide block copolymers, such as the products sold under the trade name Pluronic® by the company BASF AG (for example, Pluronic® F68, F127, L64 or L61) or the products sold under the trade name Synperonic® by the company Unichema Chemie BV (for example Synperonic® PE/F68, PE/L61 or PE/L64).

The surfactant layer located at the periphery of the oil droplets of the emulsion of the invention may also comprise at least one agent for targeting a biological activity of interest, said targeting agent being made up of an amphiphilic grafting cosurfactant of which the hydrophilic part is covalently bonded to a biological ligand. The presence of a targeting agent makes it possible to target a biological process of particular interest.

According to one advantageous embodiment of the invention, said targeting agents are chosen from the compounds of formula (I) below:

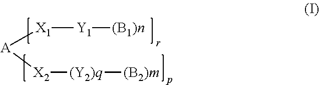

in which:
A is the lipophilic part of an amphiphilic grafting cosurfactant (CoTA),
X₁ and X₂, which may be identical or different, constitute the hydrophilic part of said cosurfactant CoTA and are composed of a flexible spacer arm chosen from saturated or unsaturated, linear or branched carbon-based chains optionally substituted, interrupted and/or terminated with one or more heteroatoms chosen, for example, from N, O, P and S, and/or with one or more groups chosen, for example, from C₁-C₄ alkyl, C₁-C₄ alkoxy or aryl radicals, or with one or more functions chosen from ether, ester, amide, carbonyl, carbamate, urea, thiourea and disulphide functions;
Y₁ and Y₂, which may be identical or different, are chosen from chemical groups capable of linking X₁ and B₁, respectively X₂ and B₂, by covalent bonds;
B₁ and B₂, which may be identical or different, are biological ligands, one of the ends of which is involved in the covalent bond formed with X₁, respectively X₂;
n is an integer between 1 and 20, limits inclusive;
q is an integer equal to 0 or 1;
m is an integer between 0 and 20, limits inclusive, it being understood that m=0 when q=0;
p is an integer between 0 and 10, limits inclusive; and

R is an integer between 0 and 10, limits inclusive.

The lipophilic part (A) of the grafting cosurfactant CoTA present in the targeting agent of formula (I) enables it to anchor itself to the surface of the oil droplets within the peripheral surfactant layer. It may be composed in particular of a saturated or unsaturated, linear or branched C₆-C₂₆ alkyl chain.

The hydrophilic part of the CoTA constituting the spacer arms X₁ and X₂ of the compounds of formula (I) above may in particular be chosen from chains made up of polyoxyethylene or dextran units.

According to one advantageous embodiment of the invention, the covalent bonds (functional groups Y₁/Y₂) providing the attachment of X₁/X₂ to the B₁/B₂ units are derived from the reaction between a chemical function initially carried by the hydrophilic part of the CoTA before its reaction with B₁/B₂, and a complementary chemical function carried by the biological ligands B₁/B₂ before the reaction thereof with X₁ respectively X₂. By way of non-limiting and non-exhaustive example, mention may be made in particular of the covalent bonds resulting from the reaction:
of an amine and of an ester that is activated, for example with an N-succinimidyl group, resulting in the formation of amide bonds;
of an oxyamine and of an aldehyde, resulting in the formation of oxime bonds; and
of an maleimide and of a thiol, resulting in the formation of thioether bonds.

Among the biological ligands that can be used as B₁/B₂ units of the targeting agents of formula (I) above, mention may be made in particular of:
i) biological ligands that make it possible to target specifically certain cells, such as peptides, for example, the RGD peptide (linear or cyclized), their derivatives and their analogues (for example: the octeotrate peptide, an analogue of somatostatin, an analogue of bombesin, neurotensin, EGF, VIP, etc.); proteins, antibodies, their derivatives or their analogues; monosaccharides such as glucose, oligosaccharides, polysaccharides, their derivatives and their analogues; oligonucleotides, DNA, their derivatives and their analogues; organic molecules such as folate, bisphosphonate pamidronate and organometallic complexes, the targeting activity of which is due to the molecular recognition of these ligands by receptors overexpressed at the surface of the cells of the region of interest;
ii) biological ligands that are labels for a given biological activity, for example, for an enzymatic activity. By way of example of such ligands, mention may, for example, be made of peptides that can be cleaved by a given protease, onto the end of which an inhibitor of the label fluorescence will be grafted. Ligands of this type make it possible to specifically image the enzymatic activity of the protease (C. H. Tung, mentioned above). Another example consists of the biological ligands comprising a disulphide bridge separating the label from an inhibitor of its fluorescence. Such a biological ligand then makes it possible to specifically image the internalization of the optical probe in a cell, as described, for example, in Patent Application FR 2 888 938.

The coupling of the biological ligands to the grafting cosurfactants CoTA can be carried out either before emulsification or after emulsification. In the latter case, it is necessary for the chemical reactions employed to be compatible with the colloidal stability of the emulsions. They should be in particular carried out in an aqueous solution at a pH that is neither too acidic nor too basic (pH 5-11).

The continuous phase of the emulsion in accordance with the invention is an aqueous phase, preferably made up of water and/or of a physiologically acceptable buffer, such as a phosphate buffer, for example PBS (phosphate buffered saline) or of a sodium chloride solution.

The emulsion in accordance with the invention may be prepared by any conventional method known to those skilled in the art for preparing emulsions, for example according to a method comprising the following steps:
a) the preparation of an oily premix for the dispersed phase of the emulsion, consisting in mixing the various biocompatible oily constituents in an organic solvent such as, for example, chloroform so as to obtain, after evaporation of the solvent, a homogeneous oily premix for the dispersed phase. When the donor fluorescent label and/or the acceptor label are lipophilic and are to be encapsulated in the oil droplets, they are added to this oily premix and homogeneously mixed. The solubilizing lipid is also added to the oily premix, either simultaneously with or separately from the labels. When the donor fluorescent label and/or the acceptor label are amphiphilic and have a sufficient solubility in the oil phase, they are integrated into this step a);
b) the preparation of the continuous phase of the emulsion by mixing, in an aqueous phase, preferably under hot conditions, at least one amphiphilic surfactant, and preferably at least one cosurfactant, in particular a stealth cosurfactant, and optionally at least one agent for targeting a biological activity of interest, said targeting agent being made up of an amphiphilic grafting cosurfactant of which the hydrophilic part is covalently bonded to a biological ligand. When the donor fluorescent label and/or the acceptor label are hydrophilic and are to be linked to the surfactant molecules, in particular the amphiphilic surfactant molecules, or are amphiphilic and have a low solubility in the oil phase and are to be linked to the surfactant molecules, they are added at this step b) of the method of preparation; and
c) the addition of the continuous phase to the dispersed phase and the emulsification of the resulting mixture until a homogeneous emulsion is obtained in which the average diameter of the oil droplets is preferably greater than 10 nm. This emulsification may, for example, be carried out using a sonicator, for a period of between 4 and 10 minutes.
   In general:
when the label or labels are lipophilic, they are added to the oily premix and will be encapsulated in the oil droplets once the emulsion has formed;
when the label or labels are hydrophilic, they are added to the aqueous continuous phase and will be linked to the surfactant molecules in the final emulsion; and
when they are amphiphilic, they are added to the oil phase or to the aqueous continuous phase, depending on the phase in which they are most soluble. When added to the oil phase because of their good solubility in this phase, if they have sufficient lipophilicity they will be encapsulated in the oil droplets of the emulsion once the emulsion has formed; otherwise, they will be linked to the membrane of the oil droplets via their lipophilic part, while their hydrophilic part will be in the surfactant layer.

According to one particular embodiment, and when the oil phase of the emulsion is composed of at least one plant or animal oil or of at least one crystallizable oil rich in C₈-C₁₈ fatty acid glycerides, the surfactant used to stabilize the emulsion can be incorporated completely or partially into the dispersed phase during step b) above. This embodiment makes it possible to prevent the formation of liposomes during the preparation of the emulsion in accordance with the invention and is particularly advantageous when said surfactant is soybean lecithin.

Before its use, the emulsion is then preferably diluted, for example, 50/50, and sterilized, for example, by filtration. This filtration step makes it possible, moreover, to eliminate the possible aggregates which might have formed during the preparation of the emulsion.

As has been amply described and explained above, the fluorescent emulsions in accordance with the invention may be used in particular for the detection of an activity of interest in vivo or in vitro.

A second subject of the present invention is therefore a labelling reagent for monitoring an activity of interest, characterized in that it comprises at least one fluorescent emulsion in accordance with the invention and as described above.

According to one particular and preferred embodiment of the invention, the reagent is an in vivo diagnostic reagent.

Finally, a subject of the invention is the use of at least one fluorescent emulsion as described above, for the preparation of a labelling reagent for monitoring an activity of interest in vivo by fluorescence imaging and in particular by time-resolved fluorescence (pulsed fluorescence) imaging and/or for aiding in the development and optimization of therapeutic tools, such as drugs. In fact, such a reagent can enable:
the detection of cancerous cells in animals, optionally in humans, by fluorescence imaging, preferably by non-invasive fluorescence imaging;
the detection of atheroma plaques in animals, optionally in humans, by fluorescence imaging, preferably by non-invasive fluorescence imaging;
the detection of β-amyloid fibres characteristic of neurodegenerative diseases in animals, optionally in humans, by fluorescence imaging, preferably by non-invasive fluorescence imaging;
the in vivo monitoring of enzymatic processes in animals, optionally in humans, by fluorescence imaging, preferably by non-invasive fluorescence imaging;
the in vivo monitoring of gene expression in animals, by fluorescence imaging, preferably by non-invasive fluorescence imaging;
the evaluation of a therapy in animals, by fluorescence imaging, preferably by non-invasive fluorescence imaging; or else
the monitoring of the biodistribution of a drug or an active principle or a substance of interest, of the controlled delivery thereof, and of the effectiveness thereof, in a host medium. The term “host medium” is understood to mean a human, an animal, a plant or any other synthetic or natural medium in which the distribution or delivery to the intended target is to be monitored.

However, the emulsion of the invention may also be used in many other applications such as, for example, for studying nanoemulsions in order to determine their properties, for example the moment when they break or the rate at which they release the labels.

When the emulsion is used for optical fluorescence imaging in human or animal tissue, the donor fluorescent label must emit and absorb in the wavelength range between 640 and 900 nm and the acceptor label must absorb in this same range. When the acceptor label is itself fluorescent, then it must also emit in this wavelength range. Again, in this case, the oil droplets preferably have an average diameter between 10 and 200 nm inclusive.

In the other cases, the emission and absorption wavelengths of the labels are, of course, to be determined according to the particular host medium.

The use of the emulsions of the invention affords many advantages.

Firstly, it makes it possible to lower the spurious signal due to the auto-fluorescence of the host medium, which occurs in the absence of a fluorescent label, by shifting the fluorescence of the acceptor label into wavelength ranges where the auto-fluorescence of the host medium is lower.

Thus, the desired signal/spurious signal ratio is improved.

Thereafter, it is possible to obtain different information, to perform multiplexing, i.e. to label certain regions differently.

To do this, a first emulsion, containing only the donor fluorescent label, and an emulsion in which the donor fluorescent label and an appropriate acceptor fluorescent label are contained will be injected. By illuminating the images with two sets of filters, it is possible to display each region where the various emulsions have accumulated. The regions where there is only fluorescence from the donor fluorescent label correspond to an accumulation of the corresponding emulsion, whereas the regions in which only fluorescence from the acceptor label is seen correspond to the regions of accumulation of the emulsion containing the donor fluorescent label and the acceptor label. The labels may be excited by the same laser in an absorption region common to the two labels, or else they may be excited at two different wavelengths so as to excite only the donor fluorescent label, on the one hand, and the acceptor label on the other. This makes it possible, in addition, to display the regions in which only the fluorescence of the acceptor label is seen. Depending on the specificity of each of the labels, the information obtained is complementary.

To monitor the delivery of a substance or a drug, it is possible, for example, to link one of the labels to the substance or drug and this label linked to the drug or to the substance is encapsulated together with the other label in the oil droplets.

When the drug (or substance) or the free label leaves the droplets, the fluorescence of the donor fluorescent label reappears.

However, for each component—drug or substance, donor fluorescent label and acceptor label—the three locations within the oil droplets, linked to the oil droplet membrane directly, or indirectly via surfactants, are possible.

For all these applications, a preferred donor fluorescent label/acceptor label pair is a DiR/ICG pair. The donor fluorescent label is 1,1′-dioctadecyl-3,3,3′,3′-tetramethylindotricarbocyanine iodide (DiR), which is lipophilic, and the acceptor label is indocyanine green (ICG), which is amphiphilic. With this pair, a high FRET efficiency of greater than 80% has been obtained.

Another particularly preferred suitable pair according to the invention for introduction into the emulsion of the invention is a DiD/DiR pair.

In this pair, the donor fluorescent label is 1,1′-dioctadecyl-3,3,3′,3′-tetramethylindodicarbocyanin perchlorate (DiD), which is lipophilic, and the acceptor label is 1,1′-dioctadecyl-3,3,3′,3′-tetramethylindotricarbocyanine iodide (DiR), which is also lipophilic.

By exciting the DiR fluorophore at 635 nm with a picosecond laser and by recording the fluorescence as a function of time, the transfer of energy from DiD to DiR has been demonstrated.

As regards the respective concentrations of the donor fluorescent label and of the acceptor label, it will be clearly apparent to those skilled in the art that these concentrations must be at least equal to each other. However, to further improve the proximity of the two labels, and also to increase the acceptor label fluorescence signals when the acceptor label is itself a fluorescent label, it is preferred, in the emulsions of the invention, to use acceptor label concentrations to 20 times higher than those of the donor fluorescent label.

1: A fluorescent emulsion, comprising:
at least one aqueous continuous phase in which droplets of at least one oil phase are dispersed;
at least one pair of labels, differing from one another, comprising a donor fluorescent label that absorbs at a wavelength λ₁ and emits at a wavelength λ₂, different from λ₁, and an acceptor label that absorbs at the emission wavelength λ₂ of the donor fluorescent label;
at least one amphiphilic surfactant; and
at least one solubilizing lipid,
wherein:
the oil phase droplets are stabilized by a surfactant layer;
the donor fluorescent label and the acceptor label are kept close together by encapsulation of one of them in the oil phase droplets and either by linking the other of them to an interface of the oil phase droplet and the aqueous phase, or by encapsulation of the other of them in the oil phase droplets; and
the emulsion is an oil-in-water emulsion.

2: The fluorescent emulsion of claim 1, wherein the acceptor label re-emits light energy emitted by the donor fluorescent label in the form of light energy having a wavelength λ₃, which differs from the wavelengths λ₁ and λ₂.

3: The fluorescent emulsion of claim 1, wherein the acceptor label re-emits no or little light energy provided by the donor label in the form of light energy.

4: The fluorescent emulsion of claim 2, wherein the wavelengths λ₁, λ₂ and λ₃ are in a range of 640 and 900 nm inclusive.

5: The fluorescent emulsion of claim 1, wherein the oil phase droplets have an average diameter in a range of 10 and 200 nm inclusive.

6: The fluorescent emulsion of claim 1, wherein the donor fluorescent label and the acceptor label
are, each independently of the other, lipophilic or amphiphilic, and
are kept close together by encapsulation in the oil phase droplets.

7: The fluorescent emulsion of claim 1, wherein either the donor label or the acceptor label is amphiphilic and tied to the interface of the oil phase droplet and the aqueous phase by being linked directly to a membrane of the oil phase droplets.

8: The fluorescent emulsion of claim 1, wherein either the donor fluorescent label or the acceptor label is tied to the interface of the oil phase droplet and the aqueous phase by linking to the at least one amphiphilic surfactant.

9: The fluorescent emulsion of claim 8, wherein the linking is by a covalent bond.

10: The fluorescent emulsion of claim 8, wherein the linking is by a disulphide bridge or a peptide bridge or a hydrazone bond.

11: The fluorescent emulsion of claim 6, wherein the donor fluorescent label is 1,1′-dioctadecyl-3,3,3′,3′,tetramethylindodicarbocyanine perchlorate (DiD) and the acceptor label is 1,1′-dioctadecyl-3,3,3′,3′-tetramethylindotricarbocyanine iodide (DiR).

12: The fluorescent emulsion of claim 6, wherein the donor fluorescent label is 1,1′-dioctadecyl-3,3,3′,3′-tetramethylindotricarbocyanine iodide and the acceptor label is indocyanine green (ICG).

13: A method of manufacturing a labelling reagent for monitoring delivering a drug or a substance of interest into a host medium, the method comprising:
combining the fluorescent emulsion of claim 1 with the drug or substance of interest, or
combining the drug or substance of interest a component of the fluorescent emulsion of claim 1 before emulsifying.

14: A labeling, comprising:
the emulsion of claim 1: and
a drug or a substance of interest encapsulated in the oil phase droplets.

15: A labelling reagent comprising:
the emulsion of claim 1; and
a drug or a substance of interest linked to either the donor fluorescent label or to the acceptor label.

16: The method of claim 13, wherein the labelling reagent is suitable for optical fluorescence imaging.

17: The fluorescent emulsion of claim 2, wherein the oil phase droplets have an average diameter in a range of 10 and 200 nm inclusive.

18: The fluorescent emulsion of claim 3, wherein the oil phase droplets have an average diameter in a range of 10 and 200 nm inclusive.

19: The fluorescent emulsion of claim 4, wherein the oil phase droplets have an average diameter in a range of 10 and 200 nm inclusive.

20: The fluorescent emulsion of claim 2, wherein the donor fluorescent label and the acceptor label
are, each independently of the other, lipophilic or amphiphilic, and
are kept close together by encapsulation in the oil phase droplets.
